(19) United States
(12) Patent Application Publication
(10) Pub. No.: US 2026/0305933 A1
(43) Pub. Date: Oct. 8, 2026

(54) DEVICE FOR DETECTING FLUORESCENCE IMAGES

(71) Applicant: Heidelberg Engineering GmbH, Heidelberg (DE)

(72) Inventor: Jörg FISCHER, Dossenheim (DE)

(73) Assignee: Heidelberg Engineering GmbH, Heidelberg (DE)

(21) Appl. No.: 19/476,721

(22) PCT Filed: Apr. 18, 2024
(86) PCT No.: PCT/EP2024/060562

§ 371 Date: Oct. 20, 2025

(30) Foreign Application Priority Data
Apr. 20, 2023 (DE) 10 2023 110 074.2

(51) Int. Cl. A61B 3/00; A61B 3/10

(52) U.S. Cl. CPC A61B 3/0008; A61B 3/0041; A61B 3/102

(57) ABSTRACT

The invention relates to a device (1) for generating and/or displaying fluorescence images, in particular fluorescence images used in ophthalmology and for diagnosing eyes, comprising a radiation device (2) which emits excitation light for the excitation of fluorescent light, and a detection device (3) with which fluorescent light generated by the excitation light can be detected, characterised in that the radiation device (2) has multiple different light sources which each emit excitation light with different wavelengths in order to generate and/or display at least one fluorescence image and/or a multicolour fluorescence image from individual fluorescence images, and/or in that the radiation device (2) has at least one light source which emits red excitation light in order to generate and/or display a multicolour fluorescence image and/or a fluorescence image.

The invention relates to a device according to the preamble of claim 1.

The designation optical coherence tomography (typically abbreviated by OCT) is understood to refer to an imaging method. Two-dimensional and three-dimensional images of light-scattering structures can be obtained using this method. In this method, light having a certain bandwidth is typically split in a beam splitter into two partial beams. The first partial beam is incident on the sample to be examined, the second partial beam passes through a reference section. The light reflected from the sample interferes with the reference beam. The sample can be examined with depth resolution, thus in the depth of the optical axis of the first partial beam, by way of signals from the interference. If the sample is also scanned flatly or laterally using the first partial beam, three-dimensional images of the sample are obtained, so-called OCT images.

A multicolor mode MC (MC for multicolor) for reflection images has already been successfully implemented in the device “Spectralis” (tradename).

In this mode, reflection images are detected line by line, namely using three different laser light sources which emit light at 486 nm, 518 nm, and 815 nm, respectively. The reflected signal is recorded and displayed in three individual images and additionally as a so-called multicolor image, wherein three channels are combined in an artificial color image.

Against this background, the invention is based on the object of specifying a device using which fluorescence images, in particular fluorescence images which are used in ophthalmology and for diagnosing eyes, can be detected and/or displayed as free of problems and as quickly as possible.

The present invention achieves the above-mentioned object by way of the features of claim 1.

According to the invention, the above-described imaging mode, namely a multicolor mode, is transferred into the fluorescence imaging, namely preferably using three different excitation light wavelengths.

Fluorescence imaging is already available using blue (BAF) and green (GAF) excitation lasers. For a multicolor autofluorescence mode (MCAF), an additional excitation laser, such as a red laser, has to be implemented.

It has been recognized according to the invention that red lasers display a good signal-to-noise ratio (SNR) and an image quality which is comparable to NIR-AF. No bleaching effect of photoreceptors occurs. The patient experiences a higher level of comfort in comparison to the use of blue and green excitation light sources, in particular lasers. No shading due to macular pigments occurs, as is known with BAF.

The red wavelength is less susceptible to effects due to lens opacity and cataracts.

Autofluorescence imaging supplies insights into pathologies on the molecular level, for example, in conjunction with intrinsic fluorophores which are potentially used in the pathological development, and can therefore potentially supply more information earlier than structural reflection imaging or OCT imaging.

The invention described here relates in particular to the simultaneous detection of fluorescence images at three (or more) excitation light wavelengths and/or the display of autofluorescence images which were detected at three different excitation light wavelengths in a false color image.

The white balancing can be carried out in such a manner that healthy retinal tissue appears in gray (similar intensity in all three channels). This type of normalization will then highlight structures which display different spectral autofluorescence properties, for example, in the range below macular pigments, in different color tones.

The following disadvantages presently exist in a clinical aspect: The autofluorescence imaging which is known up to this point in ophthalmology takes place at three excitation light wavelengths: 488 nm, 520 nm, and 785 nm. Each of these wavelengths is accompanied by disadvantages:
488 nm: Dark areas due to absorption of macular pigments, often not set off clearly from an atrophic area, a degradation of images can occur in patients with cataract, due to high scattering rates, a bleaching effect of a photo pigment changes an image during its detection.
518 nm: A degradation of images can occur in patients with cataract, due to high scattering rates, a bleaching effect of a photo pigment changes an image during its detection, partial absorption still takes place due to macular pigments.
785 nm: low autofluorescence intensity results in noisy images.

No autofluorescence mode is optimal for routine clinical imaging. There is therefore a demand for detecting three autofluorescence images in a quick pass. The technical challenge in this case is the specification and the design of a blocking filter, which suppresses the three excitation light wavelengths 486 nm, 518 nm, and 642 nm with high efficiency (10⁻⁵ or better), while as much fluorescent light as possible can be collected in the ranges 530-635 nm and >655 nm. Furthermore, a temperature drift of laser diodes has to be taken into consideration to avoid possible losses.

The MCAF mode could only be available at 768×768 pixel (HS), similarly to the MC reflection mode. However, it could advantageously be combined with a high-resolution algorithm, for example, 1536×536 pixel, which fills every second line and column during an ART mode.

The feasibility and the quality (signal strength) of red autofluorescence images (RAF) is a challenge. A suitable blocking filter (LWP530 nm with additional dip at 642 nm) has been developed and tested in the scope of this invention. The blocking filter permits quasi-simultaneous (line by line) detection of autofluorescence images using three different excitation lasers.

One essential part of the invention is the implementation of a multicolor autofluorescence mode, in which autofluorescence images can be detected simultaneously using three (or more) different excitation light wavelengths. The detected images are displayed in a false color image, where each color channel represents the signal of one excitation light wavelength. In this way, pathological structures having different autofluorescence emission behavior can be visualized and detected quickly at various excitation light wavelengths.

The fact that different pathologies display a different emission pattern at short and long excitation light wavelengths can be used according to the invention. In addition, an increase of the efficiency is possible since the MCAF imaging mode supplies information for three or more autofluorescence channels with one single image detection.

Various combinations of wavelengths are advantageously conceivable:

486 nm, 518 nm, and 642 nm:

Visualization of macular pigment distributions, visualizations of macular autofluorescence distributions independently of the macular pigments, visualization of differences of the autofluorescence emission pattern at 486/518 nm in relation to 642 nm.

470 nm, 520 nm, and 642 nm:

Additional visualization of FPF (flavoprotein fluorescence), oxidized FPF can be used as a biomarker of mitochondrial dysfunctions.

Other wavelength combinations are conceivable depending on the excitation spectra of specific fluorophores in the retina.

The following could be essential components of the device described here: A red laser module, a cleanup filter, a decoupling beam splitter, a blocking filter (LWP 530 with a dip at 642 nm). In particular for an FPF application, an embodiment having three detection channels (480-510 nm, 530-630 nm, and >650 nm) is furthermore conceivable.

In the scope of the invention, a multicolor autofluorescence mode (MCAF) is proposed which simultaneously detects three autofluorescence images and then displays three channels in a false color image. For example, macular pigments can be displayed in yellow, since green and red channels cooperate.

The relatively simple implementation of this mode in existing devices is advantageous.

The imaging mode described here uses three different laser light sources for fluorescence excitation and therefore can work selectively through the excitation light wavelength.

It is also conceivable to implement the imaging mode described here using multiple detectors (for example, one channel at 530-630 nm and one channel at 650 nm-730 nm) and to combine it with a reflection laser at 730 nm wavelength in order to reliably compensate for eye movements in a simultaneous mode: even lines AF 486 nm (SWC) and 642 nm (LWC)/odd lines: AF 518 nm (SWC) and DR reflection 730 nm (LWC).

The mode of quantitative autofluorescence (486 nm, BAF) is of interest, but is not yet established in the clinical routine. This is related to the following disadvantages: acceptance in the patients, bleaching of photo pigments (reduction of the reproducibility), and transmittance of eye media (reduction of the comparison options between patients).

These disadvantages can be overcome using red excitation light wavelengths. The MCAF mode can advantageously be used for further analyses, for example, to assess the lens turbidity (classification of a cataract).

A red laser can also be used for reflection imaging, but probably with relatively strong lens artifacts, since lens coatings are not optimally designed for 642 nm. However, red 55° reflection images could be possible in combination with a central blocking filter.

With red autofluorescence, no bleaching effect occurs and a better signal-to-noise ratio (SNR) occurs than with IRAF at 785 nm. Chemical phototoxicity is not a concern. The image quality is restricted less by cataract influences.

In multicolor autofluorescence (MCAF), one examination supplies three autofluorescence images. Relative intensity changes (color changes in the MCAF images) can potentially show the presence of different molecular fluorophores. These fluorophores can often be distinguished better by excitation than by emission bands. This could help to distinguish markers, in particular “red tomato”, from a homogeneous lipofuscin background.

### IN THE FIGURES

FIG. 1 shows a schematic representation of the transmission behavior of a blocking filter for use in a device described here,

FIGS. 2, 2a show a schematic view of the structure of the device described here,

FIG. 3 shows, on the left, a multicolor fluorescence image (MC-AF) in a large representation and, on the right, four smaller images, wherein the small image on the top left corresponds to the multicolor fluorescence image (MC-AF), the small image on the top right corresponds to a red fluorescence image (red AF), the small image on the bottom left corresponds to a green fluorescence image (green AF), and wherein the small image on the bottom right corresponds to a blue fluorescence image (blue AF), and

FIG. 4 shows a schematic representation of an exemplary embodiment of the device according to the invention, which comprises a monitor.

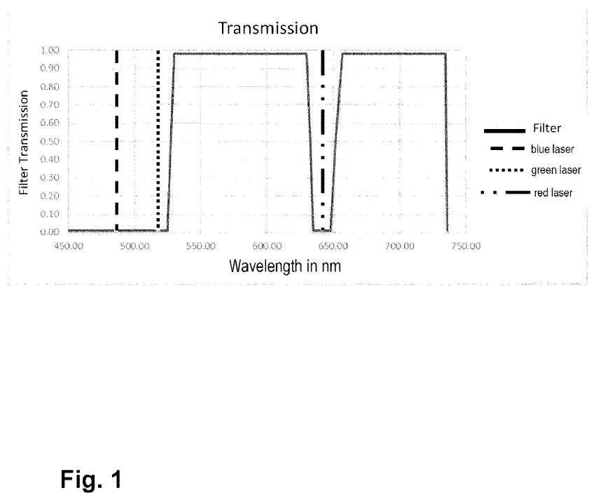

FIG. 1 shows the transmission behavior of the blocking filter on the basis of laser light wavelengths. Blue excitation lasers (BAF) and green excitation lasers (GAF) are assigned dips, very similarly as with LWP530 nm, but a further dip is provided at approximately 640 nm in order to suppress the light of the red excitation light source.

The fluorescence emission due to the red laser is detected above ≈652 nm. In principle, the fluorescence could be detected up to 900 nm or more, but in practice the OCT channel will limit the detectable emission range below the lower end of the OCT spectrum.

The imaging mode described here is advantageous to compare autofluorescence patterns of various diseases. The systematic comparison of various autofluorescence patterns could possibly show new diagnostic biomarkers for retinal diseases.

The use of the MCAF mode could be used to distinguish various specifically injected or otherwise introduced fluorophores (for example, dT tomato) in animal experiments.

Information about cataract densities could be obtained if signal strengths of autofluorescence signals are compared.

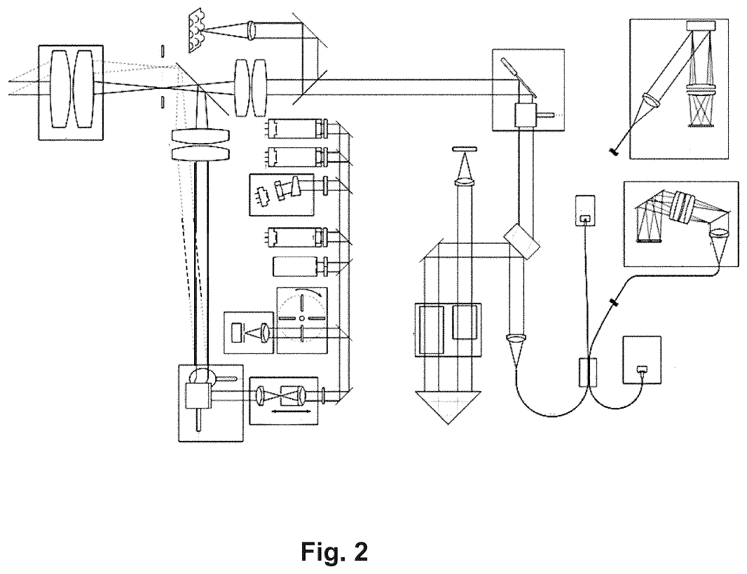

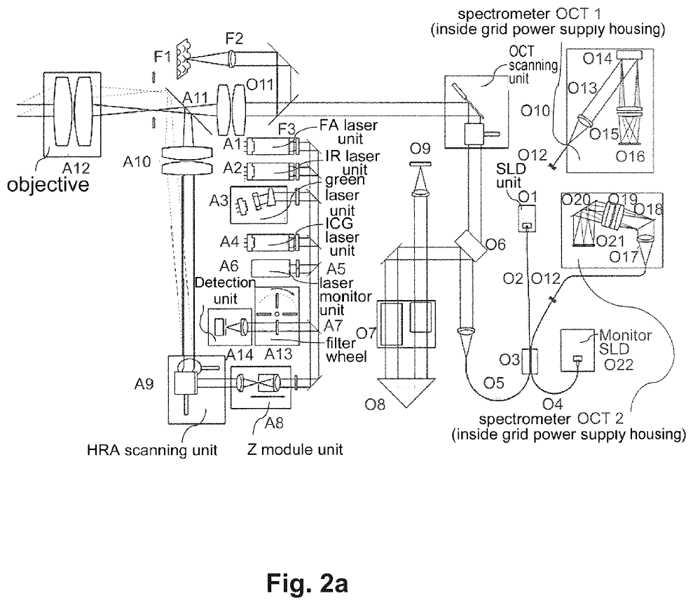

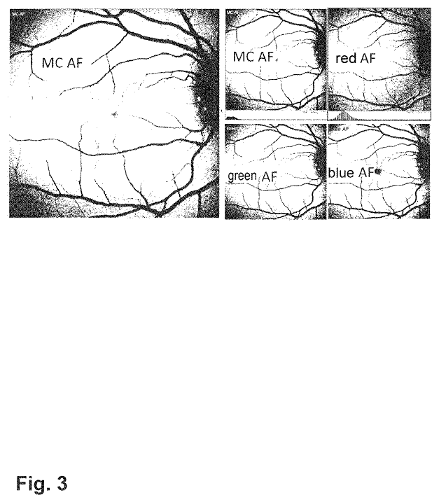

FIGS. 2 and 2a schematically show, on the basis of the product “Spectralis”, a device of the type described here. A multicolor mode MC (MC for multicolor) for reflection images has already been successfully implemented in the device “Spectralis” (tradename). In this mode, reflection images are detected line by line, namely using three different laser light sources which emit light at 486 nm, 518 nm, and 815 nm, respectively. The reflected signal is recorded and displayed in three individual images and additionally as a so-called multicolor image, wherein three channels are combined in an artificial color image.

According to the invention, the above-described imaging mode, namely a multicolor mode, is transferred into fluorescence imaging, namely preferably using three different excitation light wavelengths.

Reference is made to the above description for further specific exemplary embodiments.

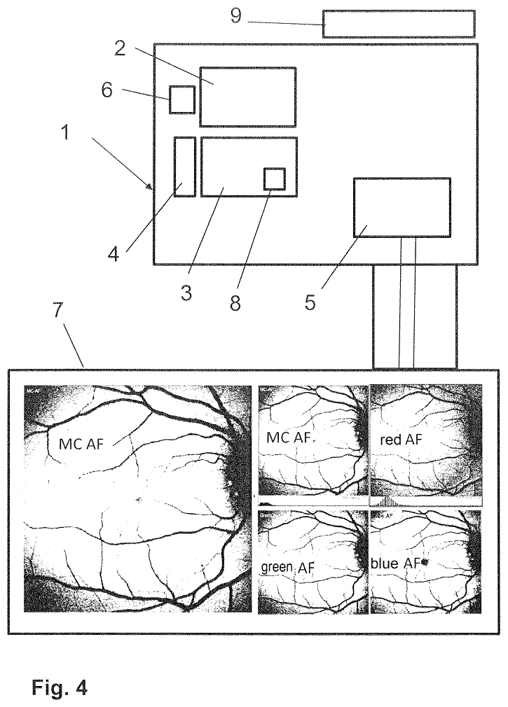

FIG. 4 schematically shows an exemplary embodiment of a device 1 for generating and/or displaying fluorescence images, in particular fluorescence images which are used in ophthalmology and for diagnosing eyes, comprising an emission unit 2, which emits excitation light to excite fluorescence light, and a detection unit 3, by which fluorescence light generated by the excitation light is detectable.

The emission unit 2 comprises multiple different light sources, which each emit excitation light at different wavelengths in order to generate and/or display at least one fluorescence image and/or a multicolor fluorescence image made up of individual fluorescence images. The emission unit 2 comprises at least one light source, which emits red excitation light in order to generate and/or display a multicolor fluorescence image and/or a fluorescence image.

The emission unit 2 emits red excitation light from the wavelength range 630 to 650 nm, preferably excitation light having a wavelength of 642 nm, wherein the detection unit 3 detects fluorescence light from the wavelength range greater than 650 nm or 650 to 900 nm or 652 to 735 nm.

The emission unit 2 emits blue excitation light from the wavelength range 470 to 490 nm, preferably excitation light having a wavelength of 486 or 488 nm, wherein the detection unit 3 detects fluorescence light from the wavelength range 480 to 510 nm or 530 nm to 630 nm or 500 to 720 nm.

The emission unit 2 emits green excitation light from the wavelength range 500 to 530 nm, preferably excitation light having a wavelength of 518 or 520 nm, wherein the detection unit 3 detects fluorescence light from the wavelength range 500 to 720 nm, 530-630 nm, or 530 to 720 nm.

The emission unit 2 comprises a first light source designed as a laser which emits blue excitation light, wherein the emission unit 2 comprises a second light source designed as a laser which emits green excitation light, and wherein the emission unit 2 comprises a third light source designed as a laser which emits red excitation light.

To detect the fluorescence light excited by the excitation light, a blocking filter 4 is arranged in front of the detection unit 3, which blocking filter 4 is not transmissive for, in particular reflected, excitation light having specific wavelengths, namely excitation light from the wavelength ranges 450 to 530 nm, 470 to 490 nm, 500 to 530 nm, and/or 630 to 650 nm, or which blocking filter 4 suppresses the, in particular reflected, excitation light or which blocking filter 4 reduces the intensity of this, in particular reflected, excitation light upon passage through the blocking filter 4 by at least a factor of 1/10,000, so that the intensity of the, in particular reflected, excitation light which has passed through is only still 1/10,000 the intensity of the, in particular reflected, excitation light incident on the blocking filter 4.

An image generating unit 5 is provided, which generates and/or displays in each case at least one fluorescence image from the fluorescence light recorded and processed thereby, wherein the fluorescence light is excited by the respective light source.

The image generating unit 5 generates and/or displays a multicolor fluorescence image as an overall image from multiple, preferably simultaneously recorded, fluorescence images, preferably from three different-colored fluorescence images.

In the multicolor fluorescence image, individual, preferably different-colored, fluorescence images are combined, in particular after weighting by means of a color balancing algorithm.

The image generating unit 5 generates and/or displays at least one fluorescence image or an overall image made up of fluorescence images as a false color image.

A signal of a respected wavelength of the respective excitation light is assigned to each color channel of the false color image.

The three wavelengths of excitation light used individually or in combination comprise 486 nm, 518 nm, and 642 nm or 470 nm, 520 nm, and 642 nm.

The image generating unit 5 carries out white balancing in a fluorescence image or an overall image made up of fluorescence images in order to display healthy tissue in gray or in a different color tone.

The image generating unit 5 generates a fluorescence image line by line and/or column by column, wherein every second line and/or every second column is generated by means of an algorithm, preferably by means of a high-resolution algorithm for 1536 by 1536 pixels, which increases the resolution of the fluorescence image.

At least one fluorescence image is a red fluorescence image, which is generated by detecting red excited fluorescence light.

A cleaning filter is provided, which suppresses the sidebands of a red light source, in particular a red laser.

A beam splitter 6 is provided for decoupling p-polarized excitation light, in particular of a red laser.

The device 1 is switchable into a mode in which red fluorescence images can be generated and/or displayed. The device 1 is switchable into a mode in which multicolor fluorescence images can be generated and/or displayed. The device 1 is switchable into a mode in which a fluorescence image or an overall image made up of fluorescence images can be generated and/or displayed as a false color image.

The device 1 also generates reflection images in addition to the fluorescence images. The device 1 generates or generates simultaneously reflection images and fluorescence images by means of the same employed light sources.

A monitor 7 is arranged, on which a red and/or a blue and/or a green fluorescence image and/or a multicolor fluorescence image combined from the above-mentioned fluorescence images and/or a false color image of at least one of the above-mentioned images are displayable.

A monitor 7 is arranged on which reflection images are also displayable in addition to fluorescence images.

The monitor 7 is part of the device 1.

The detection unit 3 comprises a chip 8, which comprises at least one or only one detection channel for detecting the respective fluorescence light.

The chip 8 comprises a blue detection channel for detecting fluorescence light from the wavelength range 480 to 510 nm, and/or comprises a green detection channel for detecting fluorescence light from the wavelength range 530 to 560 nm or 530 to 630 nm, and/or comprises a red detection channel for detecting fluorescence light from the wavelength range greater than 650 nm or greater than 650 nm to 900 nm or 650 to 730 nm.

Eye movements can be compensated during the fluorescence image generation. Even lines of the fluorescence image are generated by means of blue fluorescence light and red fluorescence light, in particular of the wavelengths 486 nm and 642 nm, respectively, wherein odd lines are generated by means of green fluorescence light and a reflection laser, in particular of the wavelengths 518 nm and 730 nm, respectively.

An arrangement comprises a device 1 as claimed in any one of the preceding claims and a spectrometer 9, by means of which an optical coherence tomography (OCT) can be carried out.

### LIST OF REFERENCE SIGNS

1 device
2 emission unit
3 detection unit
4 blocking filter
5 image generating unit
6 beam splitter
7 monitor of 1
8 chip of 3
9 spectrometer

1. A device (1) for generating and/or displaying fluorescence images, in particular fluorescence images which are used in ophthalmology and/or for diagnosing eyes, comprising an emission unit (2), which emits excitation light for exciting fluorescence light, and a detection unit (3), by which fluorescence light is detectable which is generated by the excitation light,
wherein
the emission unit (2) comprises multiple different light sources, which each emit excitation light at different wavelengths in order to generate and/or display at least one fluorescence image and/or a multicolor fluorescence image made up of individual fluorescence images, and/or
the emission unit (2) comprises at least one light source, which emits red excitation light in order to generate and/or display a multicolor fluorescence image and/or a fluorescence image.

2. The device as claimed in claim 1, wherein the emission unit (2) emits red excitation light from the wavelength range 630 to 650 nm, preferably excitation light at a wavelength of 642 nm, wherein the detection unit (3) detects fluorescence light from the wavelength range greater than 650 nm or 650 to 900 nm or 652 to 735 nm.

3. The device as claimed in claim 1, wherein the emission unit (2) emits blue excitation light from the wavelength range 470 to 490 nm, preferably excitation light at a wavelength of 486 or 488 nm, wherein the detection unit (3) detects fluorescence light from the wavelength range 480 to 510 nm or 530 nm to 630 nm or 500 to 720 nm.

4. The device as claimed in claim 1, wherein the emission unit (2) emits green excitation light from the wavelength range 500 to 530 nm, preferably excitation light at a wavelength of 518 or 520 nm, wherein the detection unit (3) detects fluorescence light from the wavelength range 500 to 720 nm, 530-630 nm, or 530 to 720 nm.

5. The device as claimed in claim 1, wherein the emission unit (2) comprises a first light source designed as a laser which emits blue excitation light, wherein the emission unit (2) comprises a second light source designed as a laser which emits green excitation light, and wherein the emission unit (2) comprises a third light source designed as a laser which emits red excitation light.

6. The device as claimed in claim 1, wherein, to detect the fluorescence light excited by the excitation light, a blocking filter (4) is arranged in front of the detection unit (3), which blocking filter (4) is not transmissive for, in particular reflected, excitation light at specific wavelengths, namely excitation light from the wavelength ranges 450 to 530 nm, 470 to 490 nm, 500 to 530 nm, and/or 630 to 650 nm, or which blocking filter (4) suppresses the, in particular reflected, excitation light, or which blocking filter (4) reduces the intensity of this, in particular reflected, excitation light upon passage through the blocking filter (4) by at least a factor of 1/10,000, so that the intensity of the, in particular reflected, excitation light which has passed through is only still 1/10,000 of the intensity of the, in particular reflected, excitation light incident on the blocking filter (4).

7. The device as claimed in claim 1, wherein an image generating unit (5) is provided, which in each case generates and/or displays at least one fluorescence image from the fluorescence light recorded and processed thereby, wherein the fluorescence light is excited by the respective light source.

8. The device as claimed in claim 7, wherein the image generating unit (5) generates and/or displays a multicolor fluorescence image as an overall image from multiple, preferably simultaneously recorded, fluorescence images, preferably from three different-colored fluorescence images.

9. The device as claimed in claim 8, wherein, in the multicolor fluorescence image, individual, preferably different-colored, fluorescence images are combined, in particular after weighting by means of a color balancing algorithm.

10. The device as claimed in claim 1, wherein that an image generating unit (5) generates and/or displays at least one fluorescence image or an overall image made up of fluorescence images as a false color image.

11. The device as claimed in claim 10, wherein a signal of a respective wavelength of the respective excitation light is assigned to each color channel of the false color image.

12. The device as claimed in claim 11, wherein the three wavelengths of excitation light used individually or in combination comprise 486 nm, 518 nm, and 642 nm or 470 nm, 520 nm, and 642 nm.

13. The device as claimed in claim 1, wherein that an image generating unit (5) carries out a white balancing in a fluorescence image or an overall image made up of fluorescence images in order to display healthy tissue in gray or in a different color tone.

14. The device as claimed in claim 1, wherein an image generating unit (5) generates a fluorescence image line by line and/or column by column, wherein every second line and/or every second column is generated by means of an algorithm, preferably by means of a high-resolution algorithm for 1536 by 1536 pixels, which increases the resolution of the fluorescence image.

15. The device as claimed in claim 1, wherein at least one fluorescence image is a red fluorescence image, which is generated by detecting red excited fluorescence light.

16. The device as claimed in claim 15, wherein a cleaning filter is provided, which suppresses the sidebands of a red light source, in particular a red laser.

17. The device as claimed in claim 1, wherein a beam splitter (6) is provided for decoupling p-polarized excitation light, in particular of a red laser.

18. The device as claimed in claim 1, wherein the device (1) is switchable into a mode in which red fluorescence images can be generated and/or displayed, and/or the device (1) is switchable into a mode in which multicolor fluorescence images can be generated and/or displayed, and/or the device (1) is switchable into a mode in which a fluorescence image or an overall image made up of fluorescence images can be generated and/or displayed as a false color image.

19. The device as claimed in claim 1, wherein the device (1) also generates reflection images in addition to the fluorescence images, and/or the device (1) generates or simultaneously generates reflection images and fluorescence images by means of the same employed light sources.

20. The device as claimed in claim 1, wherein a monitor (7) is arranged, on which a red and/or a blue and/or a green fluorescence image and/or a multicolor fluorescence image combined from the above-mentioned fluorescence images and/or a false color image of at least one of the above-mentioned images are displayable.

21. The device as claimed in claim 20, wherein a monitor (7) is arranged, on which reflection images are also displayable in addition to fluorescence images.

22. The device as claimed in claim 1, wherein the detection unit (3) comprises a chip (8), which comprises at least one or only one detection channel for detecting the respective fluorescence light.

23. The device as claimed in claim 22, wherein the chip (8) comprises a blue detection channel for detecting fluorescence light from the wavelength range 480 to 510 nm and/or comprises a green detection channel for detecting fluorescence light from the wavelength range 530 to 560 nm or 530 to 630 nm, and/or comprises a red detection channel for detecting fluorescence light from the wavelength range greater than 650 nm or greater than 650 nm to 900 nm or 650 to 730 nm.

24. The device as claimed in claim 1, wherein eye movements during the fluorescence image generation can be compensated and/or even lines of the fluorescence image are generated by means of blue fluorescence light and red fluorescence light, in particular of the wavelengths 486 nm and 642 nm, respectively, wherein odd lines are generated by means of green fluorescence light and a reflection laser, in particular of the wavelengths 518 nm and 730 nm, respectively.

25. An arrangement, comprising a device (1) as claimed in claim 1 and a spectrometer (9), by means of which an optical coherence tomography (OCT) can be carried out.
